(19) United States
(12) Patent Application Publication
(10) Pub. No.: US 2018/0372727 A1
(43) Pub. Date: Dec. 27, 2018

(54) METHODS AND KITS FOR PREDICTING THE RISK OF RELAPSE IN PATIENTS SUFFERING FROM IDIOPATHIC NEPHROTIC SYNDROME

(71) Applicants: INSERM (INSTITUT NATIONAL DE LA SANTÉ ET DE LA RECHERCHE MÉDICALE), Paris (FR); UNIVERSITÉ PARIS-EST CRÉTEIL VAL DE MARNE, Créteil (FR); ASSISTANCE PUBLIQUE-HÔPITAUX DE PARIS (APHP), Paris (FR)

(72) Inventors: Dijillali SAHALI, Creteil (FR); André PAWLAK, Creteil (FR); Mario OLLERO, Creteil (FR)

(21) Appl. No.: 16/119,130

(22) Filed: Aug. 31, 2018

Related U.S. Application Data
(63) Continuation-in-part of application No. PCT/EP2017/054781, filed on Mar. 1, 2017.

(30) Foreign Application Priority Data
Mar. 2, 2016 (EP) 16305241.8

(51) Int. Cl. G01N 33/50

(52) U.S. Cl. CPC G01N 33/5091; G01N 2800/347; G01N 2800/54

(57) ABSTRACT

The present invention relates to methods and kits for predicting the risk of relapse in patients suffering from idiopathic nephrotic syndrome. No test exists for mechanistically classifying idiopathic and secondary nor for predicting the risk of relapse, with consequent non-specific and toxic treatment regimes. In particular, the present invention relates to a method of predicting the risk of relapse in a patient suffering from idiopathic nephrotic syndrome i) comprising quantifying the level of FoxP3 positive cells and the level of CMIP positive cells in a blood sample obtained from the patient, ii) comparing the level quantified at step i) with their respective predetermined reference values and iii) concluding that the patient is at risk of relapse when the level of FoxP3 positive cells is lower than its predetermined reference value and the level of CMIP positive cells is higher than its predetermined reference value.

### FIELD OF THE INVENTION

The present invention relates to methods and kits for predicting the risk of relapse in patients suffering from idiopathic nephrotic syndrome.

### BACKGROUND OF THE INVENTION

Idiopathic nephrotic syndrome (INS) is a kidney disease defined by massive proteinuria and hypoalbuminaemia. INS is recognized as a common chronic illness in childhood. Primary INS includes two major histological variants: minimal-change nephrotic syndrome (MCNS) and focal segmental glomerulosclerosis (FSGS), which account for 70% and 20% of INS, respectively, in children, and 25% each in adults. The hallmark of MCNS is the absence of inflammatory injuries or immune complex deposits in the glomeruli, whereas FSGS is characterized by adhesion of the glomerular tuft to Bowman's capsule. Ultrastructural analysis shows glomerular morphological changes in the form of foot processes effacement.

Several decades ago, it was suggested that MCNS is a systemic disorder of T-cell function and cell-mediated immunity (1). This hypothesis was supported by several clinical observations such as the rapid occurrence of relapses upon antigen challenge (infections or immunization), hyporesponsiveness of lymphocytes to mitogens, and decreased delayed hypersensitivity (2). Allergic manifestations such as contact dermatitis, rhinitis, and asthma might be observed, particularly in children, but they are uncommon in adult patients with MCNS. However, despite the increased frequency of allergic diseases, the incidence of MCNS is remarkably stable, thus excluding close relationships between these diseases. Intriguingly, MCNS is the most common kidney disease associated with primary immunological disorders such as Hodgkin's lymphoma, leukemia, and thymoma (3-5). The sensitivity to steroids and immunosuppressive drugs is the most important clinical argument suggesting the immune origin of MCNS. Rituximab, a chimeric monoclonal antibody inhibiting CD20-mediated B-cell proliferation and differentiation, has recently gained attention as a potentially successful therapy in frequent MCNS relapsers (6). The mechanism by which Rituximab induces remission is unknown but it may involve a defect in T-B cell cooperation. Several T-cell populations are expanded during the active phase of the disease, such as CD4+ T-cells expressing the CD25 antigen (IL-2 receptor a chain) (1) and CD8+ T-cells expressing the memory T cell marker, CD45RO (7). However, the molecular link between immune disorders and the nephrotic syndrome remains elusive.

Although immune cell disorders, which may involve both innate and adaptive immunity, appear to play a role in the pathogenesis of steroid sensitive MCNS, the mechanisms by which they induce podocyte dysfunction remain unresolved. It was postulated that podocyte injury results from a circulating factor secreted by abnormal T-cells, but the possibility that the bipolarity of the disease results from a functional disorder shared by both cell systems is not excluded. MCNS relapses are associated with an activation of the immune system, including an expansion of T and B cell compartments and production of growth factors as well as many cytokines. Dysfunction of T cells is supported by three main findings: (i) inhibition of a type III hypersensitivity reaction (2); (ii) defects in immunoglobulin switch (8); (iii) unclassical T helper polarization resulting from transcriptional interference between Th1 and Th2 transcriptional factors (9).

Understanding the pathophysiology of acquired INS has become an important issue. Despite recent advances in the molecular characterization of genetic defects of several inherited causes of nephrotic syndromes, the pathophysiology of acquired INS, including MCNS and FSGS with relapse, remains unknown. These diseases have a lifelong course and often expose to drug side effects, in particular with corticosteroids and cyclosporine. No test exists for mechanistically classifying idiopathic and secondary nor for predicting the risk of relapse, with consequent non-specific and toxic treatment regimes.

### SUMMARY OF THE INVENTION

The present invention relates to methods and kits for predicting the risk of relapse in patients suffering from idiopathic nephrotic syndrome, and methods of treating patients at risk of relapse. In particular, the present invention is defined by the claims.

### DETAILED DESCRIPTION OF THE INVENTION

Clinical and experimental observations suggest that idiopathic nephrotic syndrome with relapse (INS) results from T-cell dysfunction, but the pathophysiological mechanisms remain unclear. In a previous study in patients with INS, the inventors found that CMIP is upregulated in a subset of T-cells not yet phenotypically characterized. In a recent double-blind randomized study, the inventors found that relapse were invariably associated with a profound downregulation of T regulatory cells (CD4+CD25^{high}FoxP3^{high}), concomitantly with an increase of CMIP abundance. They found that patients who developed idiopathic nephrotic syndrome in the context of inactivating FoxP3 mutation display high CMIP abundance in both T-cells and podocytes. Interestingly, in a model of FoxP3-deficient mice, they found that CMIP is exclusively upregulated in a non-functional Treg subset. Overexpression of CMIP in T-cell by targeted transgenesis resulted in increased expression of FoxP3, while its ablation by conditional knockout induced a significant reduction of FoxP3, suggesting that CMIP is an upstream actor in FoxP3 activating pathway. Collectively, these data point out the role of abnormal Treg cells overexpressing CMIP in immune disorders associated with INS. Development of a screening test in patients during the relapse and remission phases of INS, by combining FoxP3 and CMIP as markers is thus highly relevant and desirable.

The present invention relates to a method of predicting the risk of relapse in a patient suffering from idiopathic nephrotic syndrome i) comprising quantifying the level of FoxP3 positive cells and the level of CMIP positive cells in a blood sample obtained from the patient, ii) comparing the level quantified at step i) with their respective predetermined reference values and iii) concluding that the patient is at risk of relapse when the level of FoxP3 positive cells is lower than its predetermined reference value and the level of CMIP positive cells is higher than its predetermined reference value. If the patient is at risk of relapse, suitable treatments are administered. Suitable treatments include but are not limited to various immunosuppressive drugs, corticosteroids and B cell depleting agents, as listed elsewhere herein.

As used herein, the term “idiopathic nephrotic syndrome” has its general meaning in the art and is characterized by massive proteinuria due to the damage caused to the glomerular basement membrane, the main filtering unit of the kidneys. The damage is mainly associated with podocyte dysfunction resulting from either primary immune involvement of the kidney or secondary involvement due to immune mediated systemic disorders. Idiopathic nephrotic syndrome defines two main primary glomerular diseases, namely minimal change nephrotic syndrome (MCNS) and focal segmental glomerulosclerosis (FSGS). However, both histological forms may occur in the setting of haematological diseases, neoplasia or metabolic disorders and are considered as paraneoplastic or secondary causes of INS.

As used herein, the term “Risk” in the context of the present invention, relates to the probability that an event will occur over a specific time period, as in the conversion to relapse, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion. “Risk evaluation,” or “evaluation of risk” in the context of the present invention encompasses making a prediction of the probability, odds, or likelihood that an event or disease state may occur, the rate of occurrence of the event or conversion from one disease state to another, i.e., from a normal condition to relapse or to one at risk of developing relapse. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion to relapse, thus diagnosing and defining the risk spectrum of a category of subjects defined as being at risk of having relapse. In the categorical scenario, the invention can be used to discriminate between normal and other subject cohorts at higher risk of having relapse. In some embodiments, the present invention may be used so as to discriminate those at risk of having relapse from normal, or those having relapse disease from normal.

As used herein, the term “relapse” refers to the return of signs and symptoms of a disease after a patient has enjoyed a remission after a treatment. Thus, if initially the target disease is alleviated or healed, or progression of the disease was halted or slowed down, and subsequently the disease or one or more characteristics of the disease return, the patient is referred to as being “relapsed.”

In some embodiments, the method of the present invention is particularly suitable for predicting the risk of relapse when the patient was or is treated with a least one agent selected from the group consisting of immunosuppressive drugs, corticosteroids and B cell depleting agents.

### Impact of Determining the Risk of Relapse on Patient Management

Idiopathic nephrotic syndrome (INS) is often a chronic disease with relapse and remission courses. Among children and young adults, 70% exhibit frequent relapse, which are treated by high steroid dose in association with calcineurin inhibitors, mycophenolate mofetil or Rituximab. The main challenge in the management of disease is to prevent the relapse during patient follow-up. Recent investigations suggest that clinical manifestations of relapse are preceded by the dysregulation of immune system. The present test predicts the likelihood of relapse, which has significant consequences for the management such as: i) avoiding decreasing doses of steroids and/or immunosuppressive drugs in case of therapeutic withdrawal; ii) in a patient free of therapy, the reintroduction of low doses of steroids may be enough; iii) in some patients with frequent and highly steroid-dependent relapses, it would be legitimate to anticipate a new injection of rituximab if the last date more than six months and iv) in patients receiving steroids alone, it could be useful in some case to introduce another drug such as calcineurin inhibitor or mycophenolate mofetil. Therefore, the availability of a predictive test for relapse dramatically improves the management of the treatment of patients with INS. In addition, relapse may be associated with comorbidities such as thrombosis and pulmonary embolism, which can be prevented if the diagnosis is made prior to clinical manifestations.

As used herein, the term “immunosuppressive drug” refers to any substance capable of producing an immunosuppressive effect, e.g., the prevention or diminution of the immune response. Immunosuppressive drugs include, without limitation thiopurine drugs such as azathioprine (AZA) and metabolites thereof; nucleoside triphosphate inhibitors such as mycophenolic acid (Cellcept) and its derivative (Myfortic); derivatives thereof; prodrugs thereof; and combinations thereof. In some embodiments the immunosuppressive drug is ciclosporin (also named “ciclosporin” A or “CyA”) that is a competitive calcineurin inhibitor with potent immunosuppressive properties.

As used, the term “corticosteroids” has its general meaning in the art and refers to class of active ingredients having a hydrogenated cyclopentoperhydrophenanthrene ring system endowed with an anti-inflammatory activity. Corticosteroid drugs typically include cortisone, cortisol, hydrocortisone (11β,17-dihydroxy, 21-(phosphonooxy)-pregn-4-ene, 3,20-dione disodium), dihydroxycortisone, dexamethasone (21-(acetyloxy)-9-fluoro-1β,17-dihydroxy-16a-m-ethylpregna-1,4-diene-3,20-dione), and highly derivatized steroid drugs such as beconase (beclomethasone dipropionate, which is 9-chloro-11-β, 17,21, trihydroxy-16β-methylpregna-1,4 diene-3,20-dione 17,21-dipropionate). Other examples of corticosteroids include flunisolide, prednisone, prednisolone, methylprednisolone, triamcinolone, deflazacort and betamethasone. corticosteroids, for example, cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinolone, fluocinonide, betamethasone dipropionate, betamethasone valerate, desonide, desoximetasone, fluocinolone, triamcinolone, triamcinolone acetonide, clobetasol propionate, and dexamethasone.

As used herein, the term “B cell depleting agent” refers to any agent that is capable of triggering lymphodepletion of B cells. In some embodiments, the B cell depleting agent is an antibody having specificity for CD20. Examples of antibodies having specificity for CD20 include: “C2B8” which is now called “Rituximab” (“RITUXAN®”) (U.S. Pat. No. 5,736,137, expressly incorporated herein by reference), a chimaeric pan-B antibody targeting CD20; the yttrium-[90]-labeled 2B8 murine antibody designated “Y2B8” or “Ibritumomab Tiuxetan” ZEVALIN® (U.S. Pat. No. 5,736,137, expressly incorporated herein by reference), a murine IgG1 kappa mAb covalently linked to MX-DTPA for chelating to yttrium-[90]; murine IgG2a “BI,” also called “Tositumomab,” optionally labeled with radioactive 1311 to generate the “1311-B1” antibody (iodine 131 tositumomab, BEXXAR™) (U.S. Pat. No. 5,595,721, expressly incorporated herein by reference); murine monoclonal antibody “1F5” (Press et al. Blood 69 (2):584-591 (1987) and variants thereof including “framework patched” or humanized 1F5 (WO03/002607, Leung, S.; ATCC deposit HB-96450); murine 2H7 and chimeric 2H7 antibody (U.S. Pat. No. 5,677,180, expressly incorporated herein by reference); humanized 2H7, also known as ocrelizumab (PRO-70769); Ofatumumab (Arzerra), a fully human IgG1 against a novel epitope on CD20 huMax-CD20 (Genmab, Denmark; WO2004/035607 (U.S. Ser. No. 10/687,799, expressly incorporated herein by reference)); AME-133 (ocaratuzumab; Applied Molecular Evolution), a fully-humanized and optimized IgG1 mAb against CD20; A20 antibody or variants thereof such as chimeric or humanized A20 antibody (cA20, hA20, respectively) (U.S. Ser. No. 10/366,709, expressly incorporated herein by reference, Immunomedics); and monoclonal antibodies L27, G28-2, 93-1B3, B-CI or NU-B2 available from the International Leukocyte Typing Workshop (Valentine et al, In: Leukocyte Typing III (McMichael, Ed., p. 440, Oxford University Press (1987)). Further, suitable antibodies include e.g. antibody GA101 (obinutuzumab), a third generation humanized anti-CD20-antibody of Biogen Idec/Genentech/Roche. Moreover, BLX-301 of Biolex Therapeutics, a humanized anti CD20 with optimized glycosylation or Veltuzumab (hA20), a 2nd-generation humanized antibody specific for CD20 of Immunomedics or DXL625, derivatives of veltuzumab, such as the bispecific hexavalent antibodies of IBC Pharmaceuticals (Immunomedics) which are comprised of a divalent anti-CD20 IgG of veltuzumab and a pair of stabilized dimers of Fab derived from milatuzumab, an anti-CD20 mAb enhanced with InNexus' Dynamic Cross Linking technology, of Inexus Biotechnology both are humanized anti-CD20 antibodies are suitable. Further suitable antibodies are BM-ca (a humanized antibody specific for CD20 (Int J. Oncol. 2011 February; 38(2):335-44)), C2H7 (a chimeric antibody specific for CD20 (Mol Immunol. 2008 May; 45(10):2861-8)), PRO131921 (a third generation antibody specific for CD20 developed by Genentech), Reditux (a biosimilar version of rituximab developed by Dr Reddy's), PBO-326 (a biosimilar version of rituximab developed by Probiomed), a biosimilar version of rituximab developed by Zenotech, TL-011 (a biosimilar version of rituximab developed by Teva), CMAB304 (a biosimilar version of rituximab developed by Shanghai CP Guojian), GP-2013 (a biosimilar version of rituximab developed by Sandoz (Novartis)), SAIT-101 (a biosimilar version of rituximab developed by Samsung BioLogics), a biosimilar version of rituximab developed by Intas Biophamiaceuticals, CT-P10), a biosimilar version of rituximab developed by Celltrion), a biosimilar version of rituximab developed by Biocad, Ublituximab (LFB-R603, a transgenically produced mAb targeting CD20 developed by GTC Biotherapeutics (LFB Biotechnologies)), PF-05280586 (presumed to be a biosimilar version of rituximab developed by Pfizer), Lymphomun (Bi-20, a trifimctional anti-CD20 and anti-CD3 antibody, developed by Trion Pharma), a biosimilar version of rituximab developed by Natco Pharma, a biosimilar version of rituximab developed by iBio, a biosimilar version of rituximab developed by Gedeon Richter/Stada, a biosimilar version of rituximab developed by Curaxys, a biosimilar version of rituximab developed by Coherus Biosciences/Daiichi Sankyo, a biosimilar version of rituximab developed by BioXpress, BT-D004 (a biosimilar version of rituximab developed by Protheon), AP-052 (a biosimilar version of rituximab developed by Aprogen), a biosimilar version of ofatumumab developed by BioXpress, MG-1106 (a biosimilar version of rituximab developed by Green Cross), IBI-301 (a humanized monoclonal antibody against CD20 developed by Innovent Biologics), BVX-20 (a humanized mAb against the CD20 developed by Vaccinex), 20-C2-2b (a bispecific mAb-IFNalpha that targets CD20 and human leukocyte antigen-DR (HLA-DR) developed by Immunomedics), MEDI-552 (developed by Medlmmune/AstraZeneca), the anti-CD20/streptavidin conjugates developed by NeoRx (now Poniard Pharmaceuticals), the 2nd generation anti-CD20 human antibodies developed by Favrille (now MMRGlobal), TRU-015, an antibody specific for CD20 fragment developed by Trubion/Emergent BioSolutions, as well as other precloinical approaches by various companies and entities. All aforementioned publications, references, patents and patent applications are incorporated by reference in their entireties. All antibodies disclosed in therein may be used within the present invention.

The term “blood sample” means any blood sample derived from the patient. Peripheral blood is preferred, and mononuclear cells (PBMCs) are the preferred cells. The term “PBMC” or “peripheral blood mononuclear cells” or “unfractionated PBMC”, as used herein, refers to whole PBMC, i.e. to a population of white blood cells having a round nucleus, which has not been enriched for a given sub-population. Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis which will preferentially lyse red blood cells. Such procedures are known to the expert in the art.

As used herein the term “FoxP3” has its general meaning in the art and refers to the Forkhead box P3 protein (official symbol FOXP3, Gene ID: 50943 in humans). The protein encoded by this gene is a member of the forkhead/winged-helix family of transcriptional regulators. Accordingly, the term “FoxP3 positive cell” refers to a cell that expresses FoxP3, particularly a T cell that expresses FoxP3 and more particularly a regulatory T cell that expresses FoxP3. The term “T cell”, as used herein, refers to any member of a diverse population of morphologically similar lymphocytes types that develop in the thymus and that mediate the cellular immune response of the adaptive immune system. They are characterized by the presence of a T cell receptor on the cell surface. There are several subsets of T cells, each with a distinct function (i.e. helper, memory, regulatory . . . ). As used herein, the term “regulatory T cell” embraces T cells that express CD4+CD25+FoxP3+CD127 low phenotype.

As used herein the term “CMIP” has its general meaning in the art and refers the c-maf inducing protein that is notably described by Sahali et al. ((2002) J Am Soc Nephrol 13:1238-47). The natural isoform of the human CMIP mRNA encodes a 86-kDa protein named CMIP. Accordingly, the term “CMIP positive cell” refers to a cell that expresses CMIP, particularly a T cell that expresses CMIP and more particularly a regulatory T cell that expresses CMIP.

Methods for determining the expression level of a gene are well known in the art. Most preferably, the detection and quantification of a marker that is expresses by a cell typically involve flow cytometry, Western blot and/or mRNA transcript as measured in vitro. In some embodiments, such methods comprise contacting the sample with at least one selective binding agent capable of selectively interacting with the protein of interest (i.e. FoxP3 or CMIP). The selective binding agent may be polyclonal antibody or monoclonal antibody, an antibody fragment, synthetic antibodies, or other protein-specific agents such as nucleic acid or peptide aptamers. For the detection of the antibody that makes the presence of the marker detectable by microscopy or an automated analysis system, the antibodies may be tagged directly with detectable labels such as enzymes, chromogens or fluorescent probes or indirectly detected with a secondary antibody conjugated with detectable labels. The binding agents such as antibodies or aptamers may be labelled with a detectable molecule or substance, such as preferentially a fluorescent molecule, or a radioactive molecule or any others labels known in the art. As used herein, the terms “label” and “detectable label” refer to a molecule capable of detection, including, but not limited to, radioactive isotopes, fluorescers, chemiluminescers, chromophores, enzymes, enzyme substrates, enzyme cofactors, enzyme inhibitors, chromophores, dyes, metal ions, metal sols, ligands (e.g., biotin, avidin, streptavidin or haptens), intercalating dyes and the like. The term “fluorescer” refers to a substance or a portion thereof which is capable of exhibiting fluorescence in the detectable range. Labels of interest include both directly and indirectly detectable labels. Suitable labels for use in the methods described herein include any molecule that is indirectly or directly detectable by spectroscopic, photochemical, biochemical, immunochemical, electrical, optical, chemical, or other means. Labels of interest include, but are not limited to, fluorescein and its derivatives; rhodamine and its derivatives; cyanine and its derivatives; coumarin and its derivatives; Cascade Blue and its derivatives; Lucifer Yellow and its derivatives; BODIPY and its derivatives; and the like. Labels of interest also include fluorophores, such as indocarbocyanine (C3), indodicarbocyanine (C5), Cy3, Cy3.5, Cy5, Cy5.5, Cy7, Texas Red, Pacific Blue, Oregon Green 488, Alexa fluor-355, Alexa Fluor 488, Alexa Fluor 532, Alexa Fluor 546, Alexa Fluor-555, Alexa Fluor 568, Alexa Fluor 594, Alexa Fluor 647, Alexa Fluor 660, Alexa Fluor 680, Alexa Fluor 700, JOE, Lissamine, Rhodamine Green, BODIPY, fluorescein isothiocyanate (FITC), carboxy-fluorescein (FAM), phycoerythrin, rhodamine, dichlororhodamine (dRhodamine), carboxy tetramethylrhodamine (TAMRA), carboxy-X-rhodamine (ROX), LIZ, VIC, NED, PET, SYBR, PicoGreen, RiboGreen, and the like. Fluorescent labels can be detected using a photodetector (e.g., in a flow cytometer) to detect emitted light. Enzymatic labels are typically detected by providing the enzyme with a substrate and detecting the reaction product produced by the action of the enzyme on the substrate, colorimetric labels can be detected by simply visualizing the colored label, and antigenic labels can be detected by providing an antibody (or a binding fragment thereof) that specifically binds to the antigenic label. An antibody that specifically binds to an antigenic label can be directly or indirectly detectable. For example, the antibody can be conjugated to a label moiety (e.g., a fluorophore) that provides the signal (e.g., fluorescence); the antibody can be conjugated to an enzyme (e.g., peroxidase, alkaline phosphatase, etc.) that produces a detectable product (e.g., fluorescent product) when provided with an appropriate substrate (e.g., fluorescent-tyramide, FastRed, etc.); etc. The aforementioned assays may involve the binding of the binding agents (ie. antibodies or aptamers) to a solid support. The solid surface could a microtitration plate coated with the binding partner. Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled. In a preferred embodiment, fluorescent beads are those contained in TruCount™ tubes, available from Becton Dickinson Biosciences, (San Jose, Calif.). According to the invention, methods of flow cytometry are preferred methods for measuring the level of the protein of interest (i.e. FoxP3 or CMIP). As being intra cellularly located, FoxP3 and CMIP expression may be assessed by intracellular flow cytometry using a labeled anti-Foxp3 and anti-cmip antibodies. Flow cytometry is a well-accepted tool in research that allows a user to rapidly analyze and sort components in a sample fluid. Flow cytometers use a carrier fluid (e.g., a sheath fluid) to pass the sample components, substantially one at a time, through a zone of illumination. Each sample component is illuminated by a light source, such as a laser, and light scattered by each sample component is detected and analyzed. The sample components can be separated based on their optical and other characteristics as they exit the zone of illumination. Said methods are well known in the art. For example, fluorescence activated cell sorting (FACS) may be therefore used, involves using a flow cytometer capable of simultaneous excitation and detection of multiple fluorophores, such as a BD Biosciences FACSCanto™ flow cytometer, used substantially according to the manufacturer's instructions. The cytometric systems may include a cytometric sample fluidic subsystem, as described below. In addition, the cytometric systems include a cytometer fluidically coupled to the cytometric sample fluidic subsystem. Systems of the present disclosure may include a number of additional components, such as data output devices, e.g., monitors, printers, and/or speakers, data input devices, e.g., interface ports, a mouse, a keyboard, etc., fluid handling components, power sources, etc. Intracellular flow cytometry typically involves the permeabilization and fixation of the cells (e.g. T cells). Any convenient means of permeabilizing and fixing the cells may be used in practicing the methods. For example permeabilizing agent typically include saponin, methanol, Tween® 20, Triton X-100™.

Typically, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the expression level of the selected peptide in a group of reference, one can use algorithmic analysis for the statistic treatment of the expression levels determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1−specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, it is concluded that the patient is not at risk of relapse (e.g. remission) when the level of FoxP3 positive cells is the same or is higher than its predetermined reference value and the level of CMIP positive cells is the same or lower than its predetermined reference value.

The method of the present invention is particularly suitable for determining whether a renal biopsy is required or not for confirming that the patient relapses. Renal biopsy often exposes the patients to severe complications such as severe hematuria, arterial injury, requiring sometimes arterial embolization. In children, performing renal biopsy is often difficult. So the method of the invention offers a mean to avoid the renal biopsy if it is not necessary. Indeed, when it is concluded that the diagnosis of INS is likely based on flow cytometer data, the physician can decide to avoid renal biopsy. In the opposite side, when this test is not in favor of INS disease, the physician can decide performing a renal biopsy to clarify the diagnosis.

The method of the present invention is particularly suitable for monitoring the treatment of patients suffering from idiopathic nephrotic syndrome. Accordingly, in some embodiments, the predetermined reference value is a quantification value that was previously determined. For instance, a first quantification of the FoxP3 and CMIP cells is performed during the course of the treatment and a second quantification of the same cells is performed later (after several hours, days or months). If the level of FoxP3 positive cells decreases and the level of CMIP positive cells increases between the two measurements, it is concluded that the patient would be at high relapse risk. The method of the present invention is thus particularly suitable for adjusting the treatment of the patient e.g. by adjusting the dosage, combining with administration of a new drug, substituting the current treatment by a new one. For example, if the current treatment comprises receiving one or more agents for treatment of INS, then an adjusted treatment may comprise at least one of: continuing to administer the one or more agents together with at least one additional agent that differs from the one or more agents; or administering an increased amount of at least one of the one or more agents; and/or administering new combination of agents that does not include at least one of the one or more agents. In the latter case, the new combination of agents may also include at least one additional agent that differs from at least one of the one or more agents that were previously administered, i.e. one or more of the previous agents is substituted by a different agent.

Alternatively, if the patient was previously treated with an agent for treating INS, but the treatment has been discontinued (e.g. due to success of the treatment such as disappearance or lessening of symptoms) but the test described herein indicates a risk of relapse, then one or more of the agents previously administered for treatment of INS may be administered again, or one or more agents that differ from those used in the past may be administered. Further, the patient may be monitored e.g. to determine the efficacy of the treatment, and the treatment may be adjusted as described above as needed.

As used herein, the terms “treatment” and “treat” refer to administering a therapeutically effective amount of one or more (e.g. at least one) agent suitable for treating INS. A therapeutically effective amount of a drug or drug combination is an amount that is sufficient to ameliorate, lessen or delay the onset of at least one symptom of the disease or condition and/or to avoid the development of comorbidities. In some aspects, symptoms of the disease/condition may completely abate; however, those of skill in the art will recognize that much benefit can accrue even if a complete “cure” is not attained. The amount of an agent or combination of agents and the dosage regimen is determined by a skilled medical practitioner. A dosage regimen may include continuous therapy (e.g., administering a drug at a regular intervals, e.g., at hourly intervals (e.g. every 1, 2, 3, 4, 5, 6, 7, 8, 9, or 10 hours), daily, weekly, monthly, yearly, etc.) or intermittent therapy (e.g., interrupted treatment, intermittent treatment, treatment at relapse, or treatment upon achievement of a particular predetermined criteria [e.g., disease manifestation or lack thereof, etc.]).

A further object of the invention relates to kit comprising means for performing the method of the present invention. Typically, the kit comprises means for detecting expression of FoxP3 and CMIP. In some embodiments, said means are antibodies as described above. In some embodiments, these antibodies are labelled as above described. Typically, the kits described above will also comprise one or more other containers, containing for example, wash reagents, and/or other reagents capable of quantitatively detecting the presence of bound antibodies. The kit also contains agents suitable for performing intracellular flow cytometry such as agents for permeabilization and fixation of cells. Typically compartmentalised kit includes any kit in which reagents are contained in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion. Such kits may also include a container which will accept the blood sample, a container which contains the antibody(s) used in the assay, containers which contain wash reagents (such as phosphate buffered saline, Tris-buffers, and like), and containers which contain the detection reagent.

The invention will be further illustrated by the following figures and examples. However, these examples and figures should not be interpreted in any way as limiting the scope of the present invention.

### FIGURES

FIG. 1A-C: A—Frequency of circulating Treg (CD4+CD25high FoxP3high) in patients with idiopathic nephrotic syndrome (INS) in variable phases of the disease (remission and relapse). Representative course in a patient. B—Statistical analysis from the cohort patients (p<0.001, Mann Whitney test). C—Quantitative expression of CMIP by RT-qPCR in the same patient. Note that relapse was preceded by overproduction of CMIP.

FIG. 2A-B: A—Expression of FoxP3 in CMIP-deficient mice. T-cells were isolated by negative immunomagnetic selection from spleen of CMIP conditional knockout mouse and activated as shown in left panel. FoxP3 was quantified by real time RT-qPCR at TO and one hour post-activation. B—Expression of FoxP3 in CMIP transgenic mice. CMIP was overexpressed by targeted transgenesis (in HPRT locus) under control of distal Lck promoter. Basal expression of FoxP3 in T-cells from WT and Tg mice.

FIG. 3A-B: A—Flow cytometric analysis of FoxP3 expression in CD3 CD4 CD25 bright cells. Positive cells were determined according to appropriate isotype control. Numbers indicate the percentage of positive cells. P1: analysis obtained at the inclusion, before the first infusion (placebo or Rituximab); BR: before relapse; Rel: relapse; M1-M5: post Rituximab (analyses including month-1 (M1) to month-5 (M5) after Rituximab therapy (M1-M5). (***, p<0.0001, one-way Anova test; *, p=0.03, paired test). B—Expression of CMIP transcript by real-time quantitative PCR. (*, p=0.03 Rel vs M1-M5).

### EXAMPLE 1

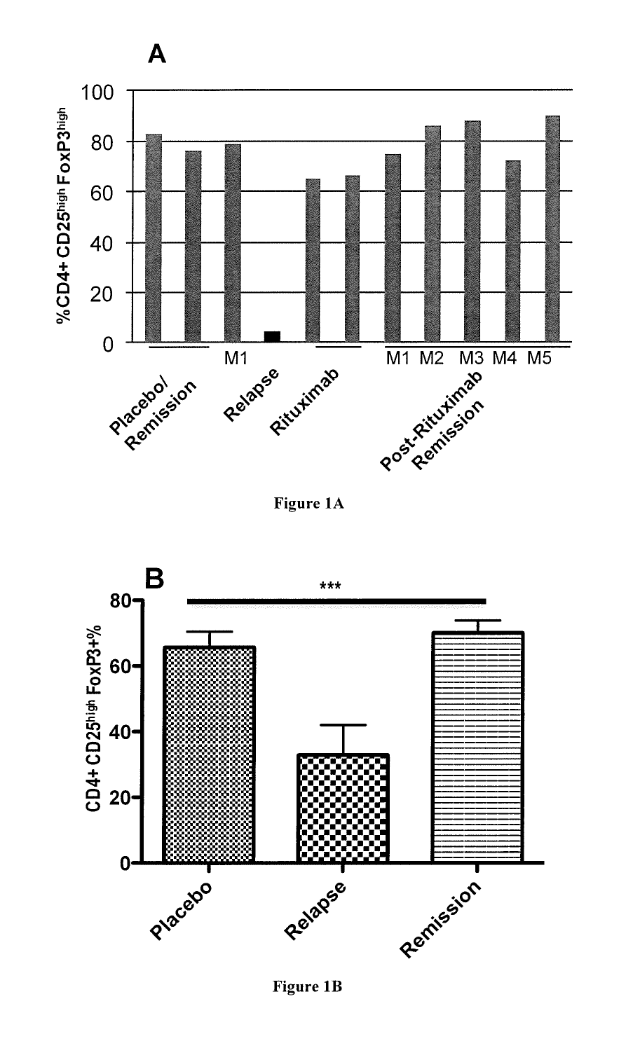

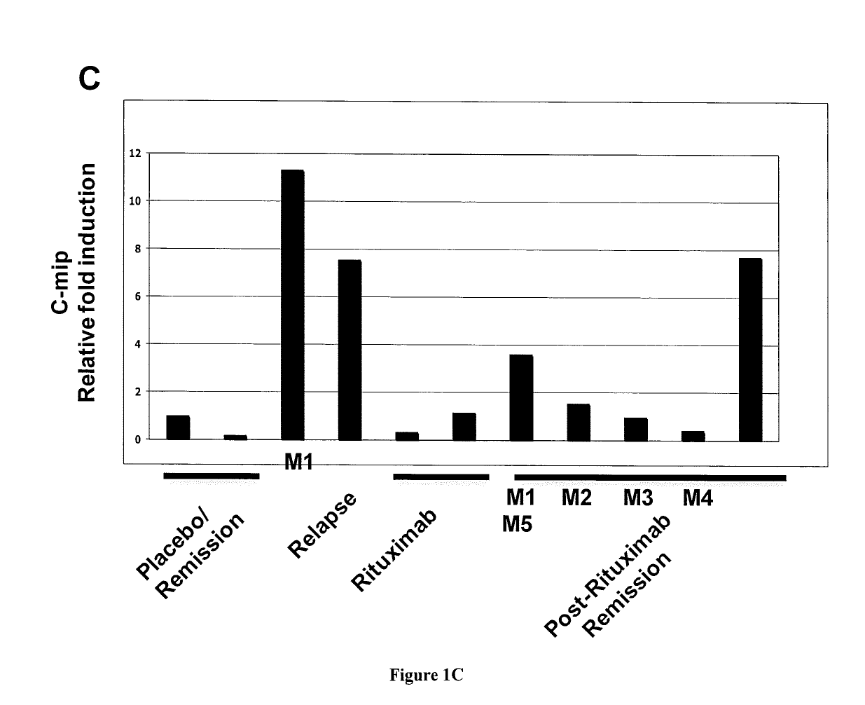

In a randomized, double-blind study aiming to understand the mechanisms underlying the effect of Rituximab on MCNS with frequent relapses, we found that relapse were associated with a significant reduction of CD4+CD25^{high} FoxP3^{high} natural T regulatory cells (nTreg) (FIGS. 1A and B). The downregulation of Treg was associated with an increase of CMIP abundance (FIG. 1C) but the potential link between these findings was unclear. Independently, we isolated T-cells by negative selection using immunomagnetic beads and analyze CMIP by confocal microscopy. We found that CMIP abundance was highly increased only in a subset of MCNS T-cells.

Several Arguments Suggest that Treg Dysfunction is Closely Associated with CMIP Induction

First to all, FoxP3 is indispensable for the differentiation, maintenance, and function of natural Treg). Scurfy mice develop an autoimmune syndrome resulting from a 2-bp insertion in the FoxP3 gene leading to production of a truncated non-functional protein The nature of the cell type in the Scurfy mouse responsible for disease induction is unknown. It has been proposed that due to the lack of FoxP3, nTreg may have altered their program from protective to autoaggressive state. Interestingly, INS has been reported in some patients with FoxP3 mutations. Although non functional, FoxP3 protein is expressed in treg of scurfy mice. Unexpectedly, CMIP is exclusively detected in this T-cell subset both in cytoplasmic and nuclear compartment and mostly colocalizes with FoxP3, whereas no c-mip expression is detected in T-cells from control mice.

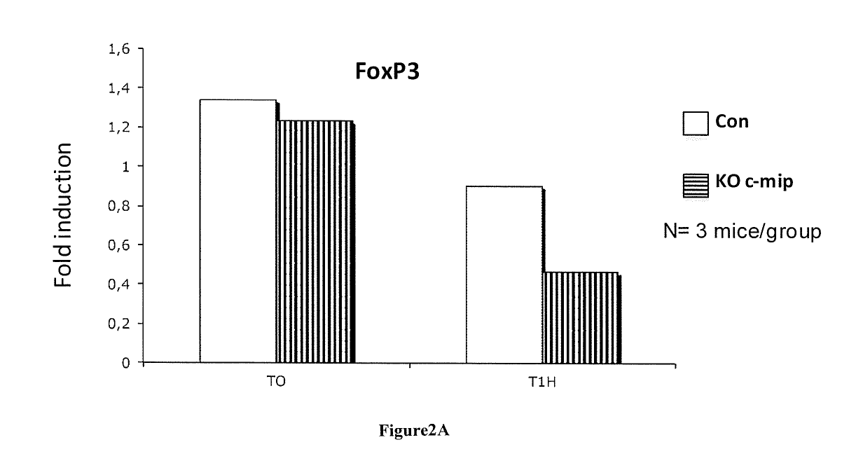

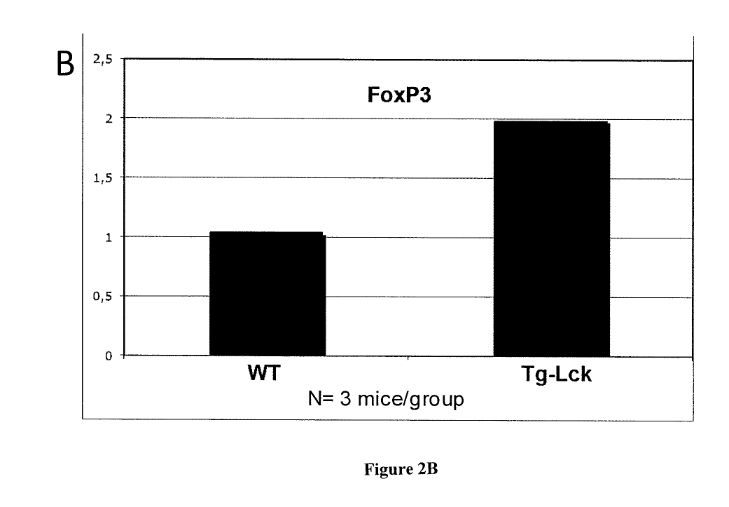

Secondly, deletion of CMIP in T-cells induces downregulation of FoxP3. We generated CMIP conditional knockout in T-cells. Analysis of FoxP3 expression by real time qPCR on c-mip-deficient T-cells isolated by immunomagnetic selection showed that the FoxP3 transcript is significant reduced upon activation by anti-CD3/CD28 (FIG. 2), suggesting that CMIP is potentially an upstream actor of the FoxP3 signaling pathway. Preliminary data indicate that CMIP-deficient mice display a lymphoproliferative phenotype currently under investigation.

Thirdly, overexpression of CMIP in T-cells by targeted transgenesis increases FoxP3 abundance and confers an anergic state. Studies on a mouse model in which CMIP was selectively overexpressed in T-cells under control of distal Lck promoter (driving CMIP expression in peripheral T-cells) showed that transgenic T-cells produce lower levels of IL-2, IFN-g, IL-4 upon anti-CD3/CD28 stimulation. Moreover, transgenic T-cell exhibits lower proliferative capacity that was restored after IL-2 addition. This “anergic state” is associated with an increase of FoxP3 abundance.

Collectively, these data point out a Treg defect in MCNS relapse. Interestingly, we observed that increase of CMIP abundance may precede of few weeks MCNS relapse. Since CMIP is scarcely or not detected in basal conditions, we postulate that it is produced in pathological situations involving a dysfunction of treg cells.

In conclusion we can conceive a screening test in patients with idiopathic nephrotic syndrome, by FACS combining Treg and CMIP as markers. This test requires the development of a monoclonal antibody targeting CMIP and FoxP3. So far, diagnosis of MCNS relies on histological analysis of renal biopsy specimen, which may expose to severe complications (severe hematuria, arterial injury, requiring sometimes arterial embolization). In children, performing kidney biopsy is often difficult. Our data suggest that peripheral monitoring of a Treg overexpressing CMIP could be a better alternative to kidney biopsy. We will evaluate whether this test could be specific for MCNS disease and discriminate between several causes of nephrotic syndrome.

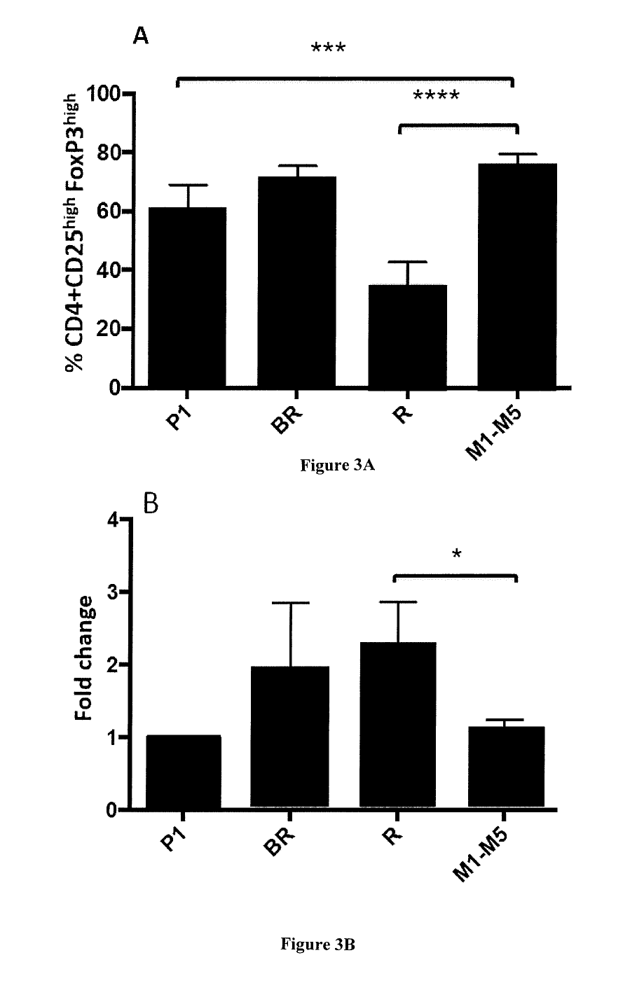

### EXAMPLE 2: DEPLETION OF CD4+CD25^{HIGH}FOXP3^{HIGH} T CELLS (TREG) IN RELAPSE (FIG. 3)

Previous studies on MCNS have suggested a defect in Treg cell population or in suppressive function, while MCNS may occur in patients with FoxP3 mutations. However, no study has documented the frequency as well as the course profile of Treg subset in the same patients before, at the time of relapse and remission phases, as well as following B-cell depletion. We found that the frequency of CD4⁺CD25^{high}FoxP3^{high} in patients receiving placebo dramatically declined in relapse (p=0.0004, one-way ANOVA, p=0.001 BR versus Rel). A significant increase and sustained Treg cell subset was observed in remission in the placebo group (p=0.0004 M1-M5 versus Rel, paired test). However, no significant difference was observed between the percentage of Treg at the inclusion (P1) and after rituximab therapy (M1-M5) in both groups (Placebo: p=0.071, P1 vs M1-M5; Rituximab: p=0.59, P1 vs M1-M5, paired test). These results suggest that MCNS relapse is associated with a Treg cell disorder. Interestingly, we found that CMIP expression increased in relapse and was downregulated in remission (*, p=0.03, paired test). Interestingly, the increase of CMIP abundance preceded nephrotic relapse by a few weeks after placebo infusion. Following remission after Rituximab therapy, CMIP was significantly reduced. Altogether these results suggest a relationship between CMIP abundance and relapse occurrence.

### REFERENCES

Throughout this application, various references describe the state of the art to which this invention pertains. The disclosures of these references are hereby incorporated by reference into the present disclosure.

1. A method of predicting the risk of relapse in a patient suffering from idiopathic nephrotic syndrome comprising i) quantifying the level of FoxP3 positive cells and the level of CMIP positive cells in a blood sample obtained from the patient, ii) comparing the levels quantified at step i) with their respective predetermined reference values iii) concluding that the patient is at risk of relapse when the level of FoxP3 positive cells is lower than its predetermined reference value and the level of CMIP positive cells is higher than its predetermined reference value, and iv) administering a therapeutically effective treatment to the patient.

2. The method of claim 1 wherein the patient suffers from minimal change nephrotic syndrome (MCNS) or focal segmental glomerulosclerosis (FSGS).

3. The method of claim 1 wherein the patient was or is treated with at least one agent selected from the group consisting of immunosuppressive drugs, corticosteroids and B cell depleting agents.

4. The method of claim 3 wherein the B cell depleting agent is an antibody having specificity for CD20.

5. The method of claim 1 wherein the blood sample is a PBMC sample.

6. The method of claim 1 wherein the FoxP3 positive cells are regulatory T cells.

7. The method of claim 1 wherein the CMIP positive cells are regulatory T cells.

8. The method of claim 1 wherein the quantification of FoxP3 positive and CMIP positive cells is performed by intracellular flow cytometry.

9. The method of claim 1 wherein it is concluded that the patient is not at risk of relapse when the level of FoxP3 positive cells is the same or is higher than its predetermined reference value and the level of CMIP positive cells is the same or lower than its predetermined reference value.

10. A method of monitoring the treatment of patients suffering from idiopathic nephrotic syndrome wherein a first quantification of the FoxP3 and CMIP cells is performed during the course of the treatment and a second quantification of the same cells is performed later wherein if the level of FoxP3 positive cells decreases and the level of CMIP positive cells increases between the two measurements, it is concluded that the patient would be at high relapse risk and administering a different treatment to the patient.

11. A kit suitable for performing the method of claim 1 comprising antibodies having specificity for CMIP and antibodies having specificity of FoxP3.

12. The method of claim 4, wherein the antibody having specificity for CD20 is rituximab.

13. The method of claim 1, wherein the therapeutically effective treatment includes administering at least one agent selected from the group consisting of immunosuppressive drugs, corticosteroids and B cell depleting agents.

14. The method of claim 10, wherein the treatment comprises receiving one or more agents for treatment for INS, and the different treatment comprises at least one of:
administering to the patient the one or more agents together with at least one additional agent that differs from the one or more agents;
administering to the patient an increased amount of at least one of the one or more agents; and/or
administering to the patient a combination of agents that does not include at least one of the one or more agents.

15. The method of claim 14, wherein the combination of agents includes at least one additional agent that differs from at least one of the one or more agents.
